(19) United States
(12) Patent Application Publication
(10) Pub. No.: US 2015/0250515 A1
(43) Pub. Date: Sep. 10, 2015

(54) SYNDESMOSIS SCREW

(71) Applicant: COORSTEK MEDICAL LLC d/b/a IMDS, Providence, UT (US)

(72) Inventors: Lance Nathan Terrill, Oviedo, FL (US); Andrew Douglas Palmer, Orlando, FL (US)

(73) Assignee: COORSTEK MEDICAL LLC d/b/a IMDS, Providence, UT (US)

(21) Appl. No.: 14/203,428

(22) Filed: Mar. 10, 2014

(51) Int. Cl. A61B 17/86

(52) U.S. Cl. CPC A61B 17/8685

(57) ABSTRACT

Disclosed bone screws include multiple screw elements engaged together to provide the required strength along a longitudinal direction and the required flexibility in other degrees of freedom. The disclosed bone screws have a low profile. Further, the bone screws allow fine-tuning of the compression to be applied to bones or joints. Instrumentation for implanting and orienting the bone screws is also disclosed herein.

### TECHNICAL FIELD

The present disclosure relates to systems, apparatuses, methods, and kits for joint repair. Specifically, this disclosure relates to syndesmosis fixation apparatuses, systems, kits, and methods suitable to accommodate or correct various patient deformities.

### BACKGROUND

A syndesmosis joint is formed by an interosseous membrane connecting the fibula to the tibia. The syndesmosis joint is a slightly movable articulation where the contiguous bony surfaces are united by the interosseous ligament. If undue stress is put on the ankle joint, the joint may fracture, with either the fibula or tibia fracturing, or possibly both. Syndesmotic injuries to the ankle occur in approximately 10% of cases of ankle fractures. However, these injuries can also occur with soft-tissue injuries in the absence of fracture. They usually result from severe external rotation of the ankle. If the syndesmosis is torn apart, the syndesmosis joint needs to be stabilized by holding the joint in position until the articulation heals. Surgeons often fix the relevant bones together with a syndesmotic screw, temporarily replacing the syndesmosis. The screw inhibits normal movement of the bones and, therefore, the corresponding joint. When the natural articulation is healed, the screw may be removed. Alternatively, surgeons use an elastic fiber wire loop tensioned and secured between metallic buttons to provide physiologic stabilization of the joint. This technique allows physiologic motion of the ankle.

However, the syndesmotic screws are prone to fracture at the fulcrum of the screw around which the fibular side of the screw rotates during gait because they are inflexible in all degrees of freedom. Further, the elastic fiber wire loop may cause an inflammatory reaction, and the buttons used in the fixation may cause soft tissue irritation. Therefore, the existing systems and procedures for syndesmosis repair may not be as effective as desired.

### SUMMARY

The present disclosure relates to bone and joint fixation, and instrumentation and methods for preparation and implantation of these devices. Joint fixation may be necessary in cases of pain and inflammation due to cartilage degeneration, nerve impingement, spinal misalignment, and motion instability. The primary examples described herein illustrate how this concept is applied to the syndesmosis joint, but this concept applies equally to other joints where similar causes of pain and inflammation are indicated. The disclosed bone screw includes a first screw element and a second screw element inserted through the first screw element and engaged therewith. Each of the first screw element and second screw element include an external thread for threaded engagement with a bone. Further, the first screw element is capable of being selectively adjusted to achieve a required compression. The bone screw when secured to the tibia and the fibula provides resistance to distraction of the syndesmosis joint. The disclosed bone screw has a low profile and is less expensive than other systems. Further, the disclosed bone screw is intrinsically stronger than a standard screw as it provides only a limited resistance to bending movement.

Those of skill in the art will recognize that the following description is merely illustrative of the principles of the disclosure, which may be applied in various ways to provide many different alternative embodiments and may be applicable outside the fields of surgery or medical devices. While the present disclosure is made in the context of syndesmosis joints for the purposes of illustrating the concepts of the design, it is contemplated that the present design and/or variations thereof may be suited to other uses, such as to support other joints in the human body and to stabilize bone fractures. Moreover, the implants, instrumentation, and methods set forth herein may be used in open, percutaneous, and/or minimally invasive procedures.

All changes that come within the meaning and range of equivalency of the claims are to be embraced within their scope.

### BRIEF DESCRIPTION OF THE DRAWINGS

Various embodiments of the present disclosure will now be discussed with reference to the appended drawings. It will be appreciated that these drawings depict only typical examples of the present disclosure and are, therefore, not to be considered limiting of its scope.

FIG. 1A is a left-side view of a bone screw in accordance with one example of the present disclosure;

FIG. 1B is a right-side view of a bone screw in accordance with another example of the present disclosure;

FIG. 1C is an isometric left-side view of the bone screw in FIG. 1A;

FIG. 1D is an isometric right-side view of the bone screw in FIG. 1B;

FIG. 2A shows a first screw element and a second screw before they are assembled together;

FIG. 2B shows a bone screw assembly after the first screw element and the second screw are assembled together;

FIG. 3A is a side view of a bone screw in accordance with another example of the present disclosure;

FIG. 3B is a cross-section view of the bone screw in FIG. 3A taken along the lines of 3B-3B of FIG. 3A;

FIG. 4 is a side view of a bone screw in accordance with another example of the present disclosure;

FIG. 5A is a side view of a second screw element of the bone screw in FIG. 1A;

FIG. 5B is a section view of the second screw element of the bone screw in FIG. 5A taken along lines 5B-5B of FIG. 5A;

FIG. 5C is an enlarged view of a portion 5C marked in FIG. 5B;

FIG. 5D is a top view of the second screw element of FIG. 5A;

FIG. 6A is an isometric view of a first screw element of the bone screw in FIG. 1A;

FIG. 6B is a top view of the first screw element in FIG. 6A;

FIG. 6C is a side view of the first screw element in FIG. 6A;

FIG. 6D is a cross-section view of the first screw element in FIG. 6C taken along lines 6D-6D of FIG. 6C;

FIG. 7A is a cross-section view along a longitudinal axis of the bone screw in FIG. 1A;

FIG. 7B is a top view of the bone screw in FIG. 7A;

FIG. 8A is a cross-section view along a longitudinal axis of a bone screw in FIG. 1B;

FIG. 8B is a top view of the bone screw in FIG. 8A;

FIG. 9A is a top view of a driver to install the bone screw in FIG. 8A;

FIG. 9B is an isometric view of the driver in FIG. 9A;

FIG. 10 shows a portion of a foot with the bone screw inserted across the syndesmosis joint according to the present disclosure; and

FIG. 11 is a flowchart illustrating a method for inserting a bone screw into the pair of bones, according to one aspect of the present disclosure.

### DETAILED DESCRIPTION

While certain embodiments are shown and described in detail below by way of illustration only, it will be clear to the person skilled in the art upon reading and understanding this disclosure that changes, modifications, and variations may be made and remain within the scope of the technology described herein. Further, while various features are grouped together in the embodiments for the purpose of streamlining the disclosure, it is appreciated that features from different embodiments may be combined to form additional embodiments which are all contemplated within the scope of the disclosed technology.

Not every feature of each embodiment is labeled in every figure where that embodiment appears, in order to keep the figures clear. Similar reference numbers (for example, those that are identical except for the first numeral) may be used to indicate similar features in different embodiments.

Any of the devices described herein may be fabricated from metals, alloys, polymers, plastics, ceramics, glasses, composite materials, or combinations thereof, including but not limited to: PEEK, titanium, titanium alloys, commercially pure titanium grade 2, ASTM F67, Nitinol, cobalt chrome, stainless steel, UHMWPE, and biodegradable materials, among others. Different materials may be used within a single part. The implants disclosed herein may also encompass a variety of surface treatments or additives to encourage bony attachment, including but not limited to: porous coatings, hydroxyapatite, TCP, anti-microbial additives, analgesics, anti-inflammatories, BMP's, PMA material, bone growth promoting material, PLLA (poly-L-lactide), PGA (polyglycolide), TCP (tricalcium phosphate), demineralized bone, cancellous bone chips, etc. Any implant disclosed herein may include a radiographic marker for imaging purposes. Any implant disclosed herein may be colored, coded, or otherwise marked to make it easier for the surgeon to identify the type and size of the implant.

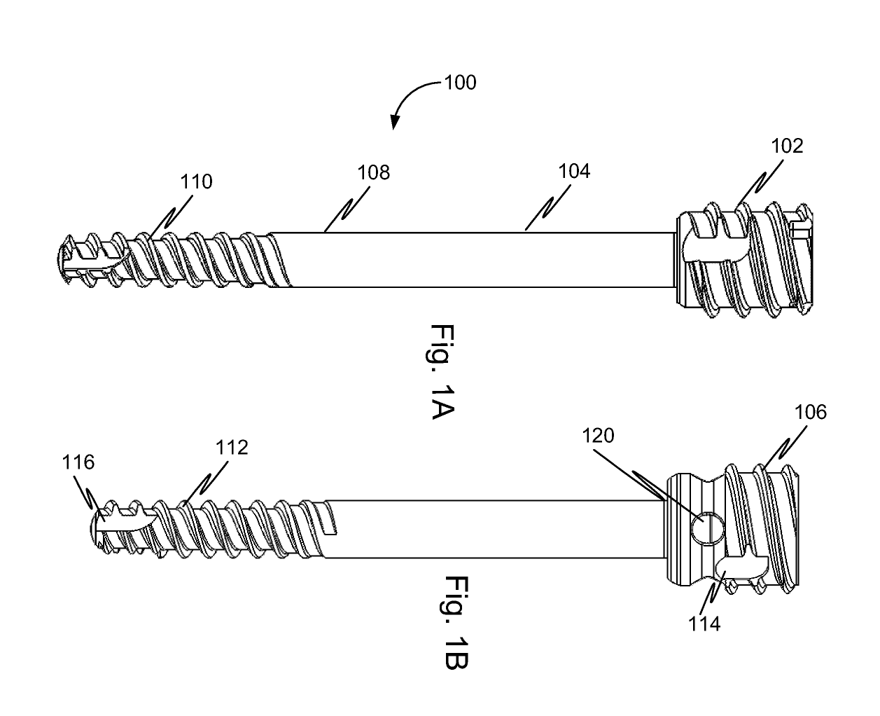

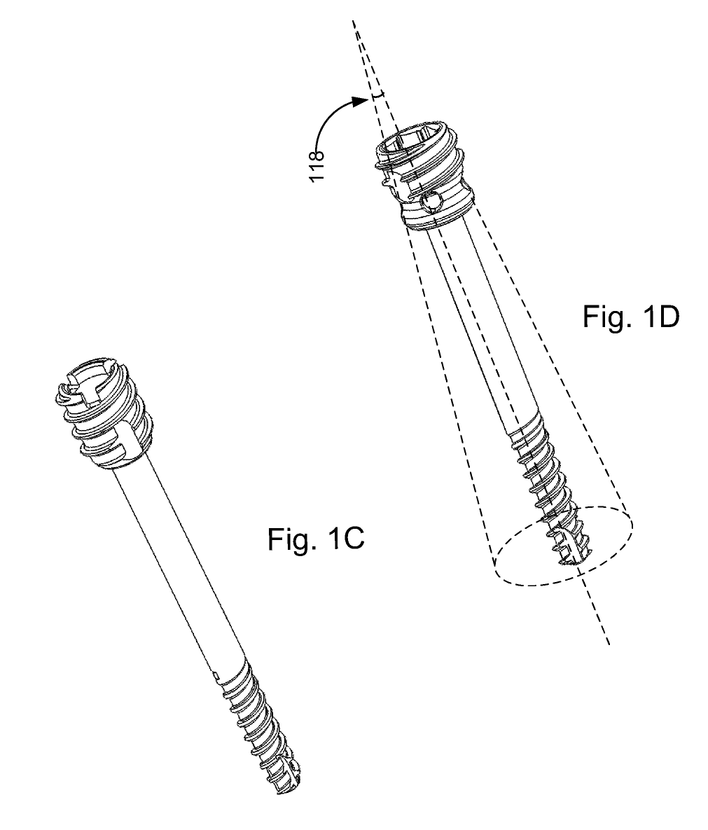

FIGS. 1A-1D illustrate one example of a bone screw 100 useful for fixing a bone fracture or a joint. The bone screw 100 includes a first screw element 102 and a second screw element 104. The first screw element 102 includes an external thread 106 with a first thread pitch. The second screw element 104 includes a shaft 108. The second screw element 104 may have a fully threaded shaft or a thread configuration with a lag (as shown in FIGS. 1A-1D). Accordingly, the shaft 108 includes a threaded portion 110. The threaded portion 110 may partially taper toward the tip of the second screw element 104. Further, the threaded portion 110 may include an external thread 112 with a second thread pitch. The external thread 106 and the external thread 112 allow the bone screw 100 to have a threaded engagement with one or more bones. When used to stabilize the syndesmosis joint, the external thread 106 engages with the fibula and the external thread 112 engages with the tibia. The first thread pitch and the second thread pitch may be the same. In another example, the first pitch and the second pitch may be different to provide compression along the screw.

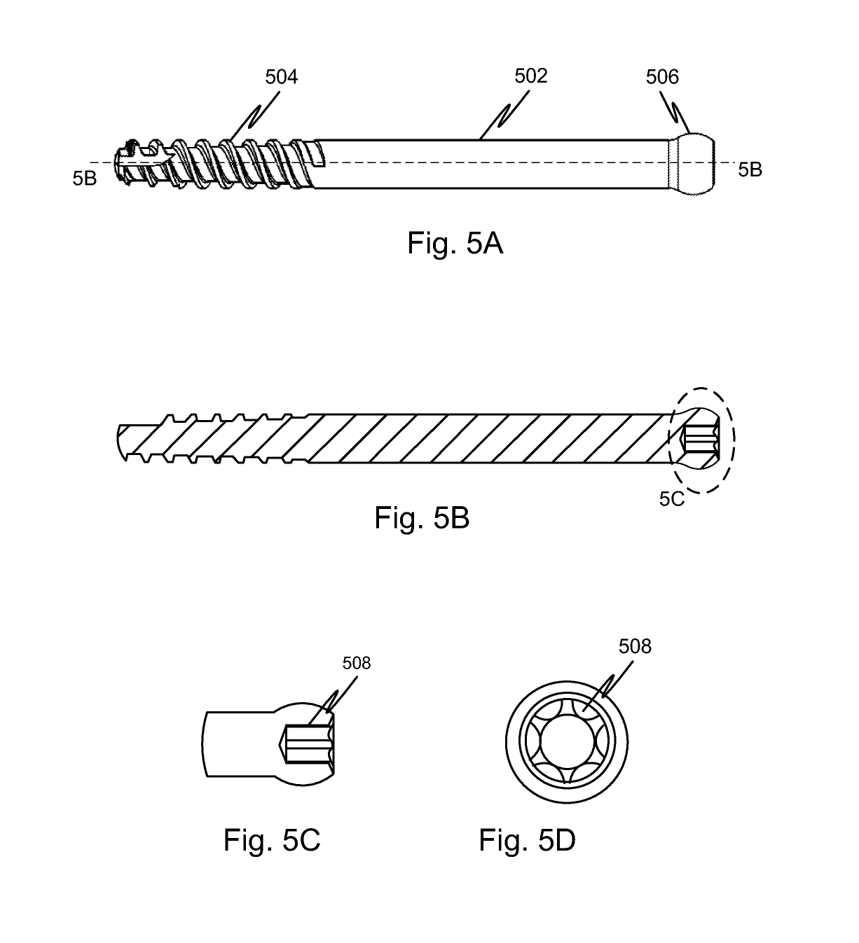

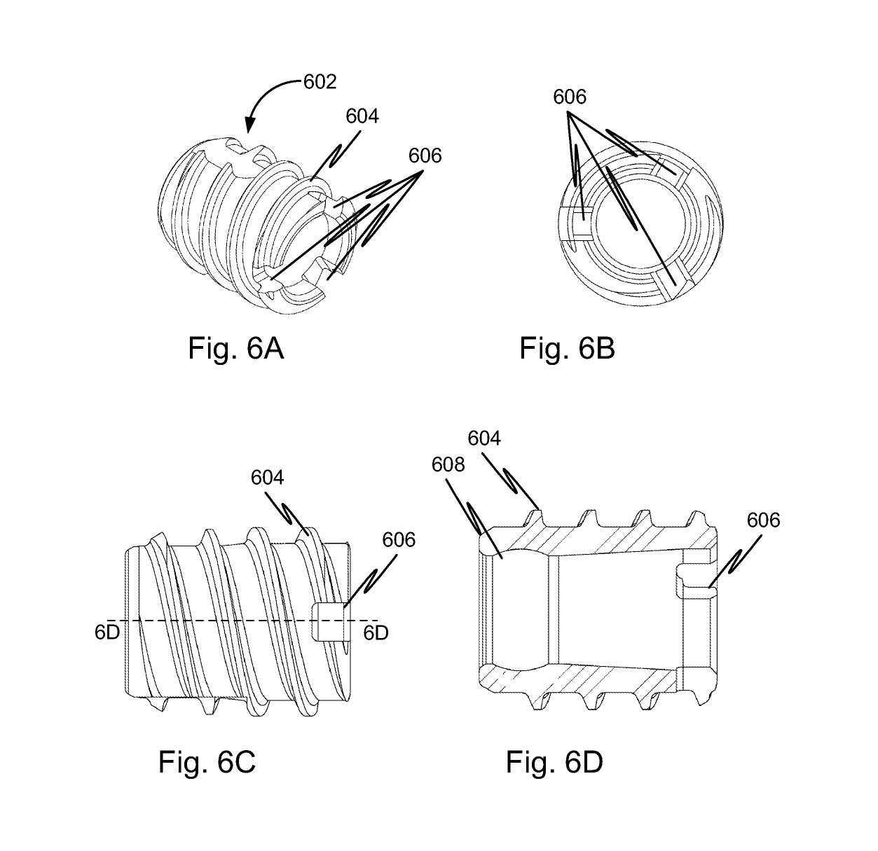

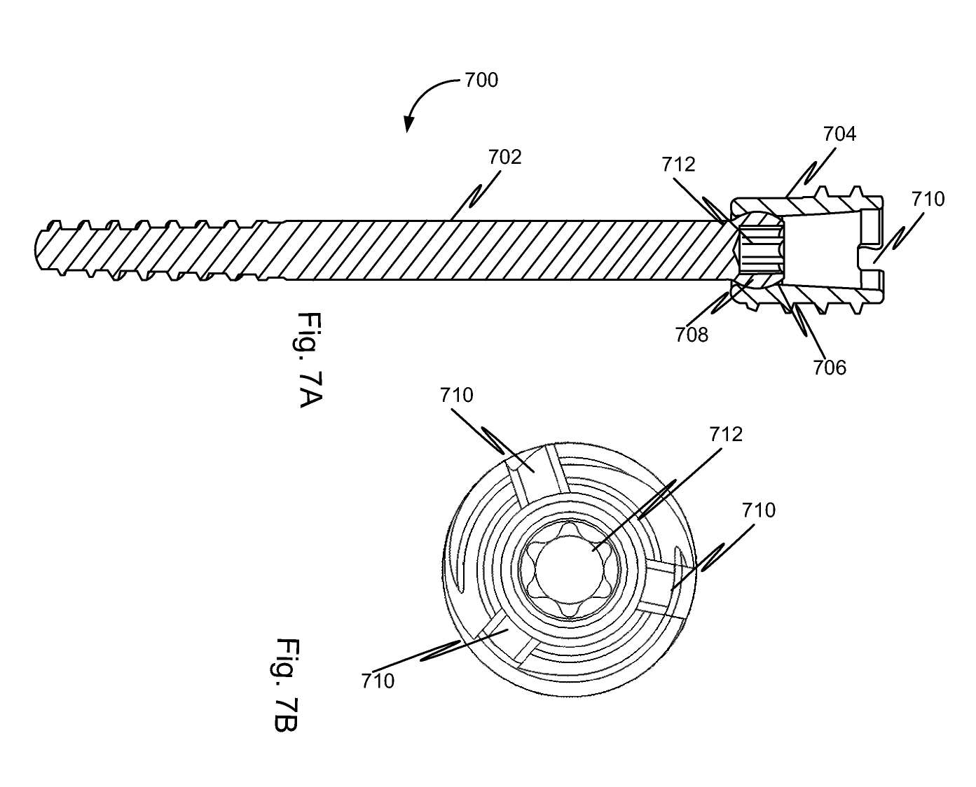

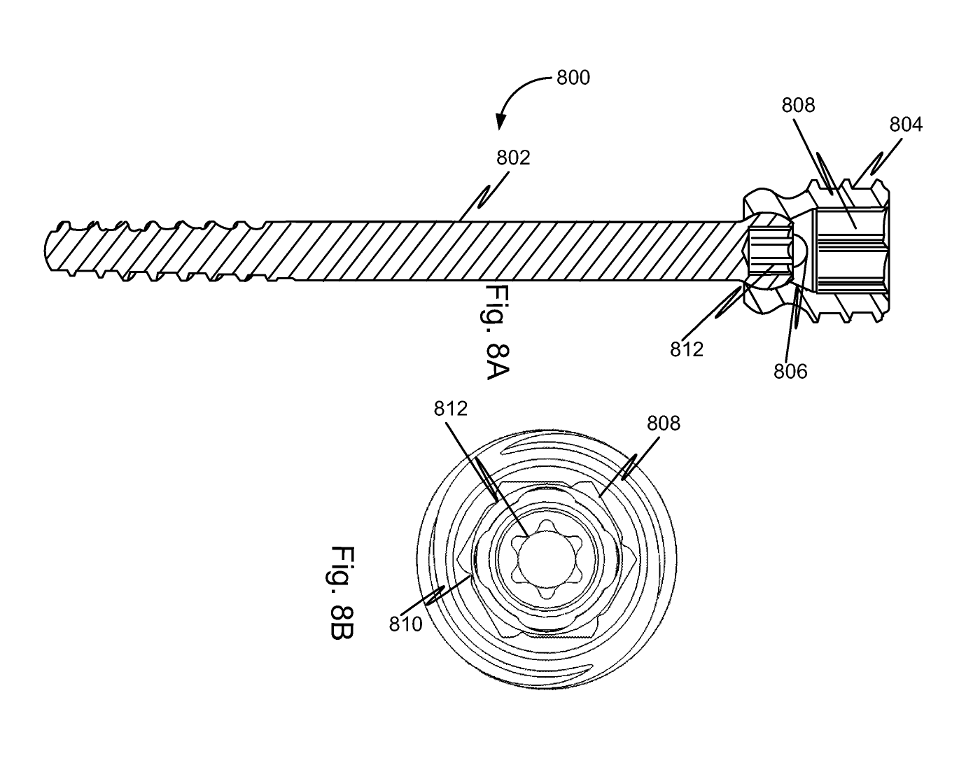

Further, the first screw element 102 includes a self-tapping feature 114 (or self-threading) to tap a drilled hole when the bone screw 100 is inserted into the one or more bones. Similarly, the second screw element 104 includes a self-tapping feature 116. Further, the second screw element 104 is cannulated down its center so that it can be placed into the one or more bones with a Kirschner wire (K-wire). The first screw element 102 and the second screw element 104 are explained in further detail in conjunction with FIGS. 6A-6D and 5A-5D below, respectively.

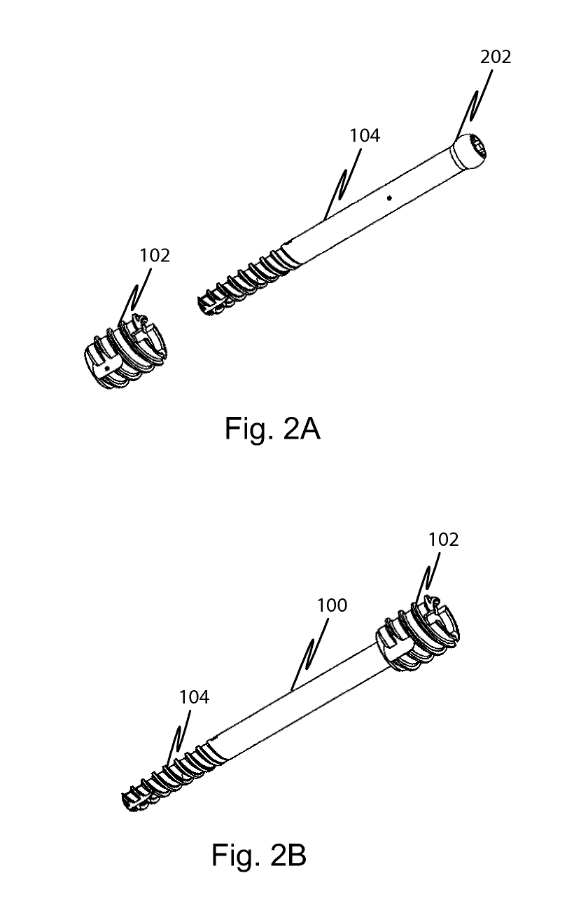

Referring now to FIG. 2A, the second screw element 104 is inserted through the first screw element 102 and engaged therewith to obtain the bone screw 100. The first screw element 102 may have a partial spherical-shaped capsule to receive a complementarily shaped partial spherical fastener head 202 of the second screw element 104. The engagement between the first screw element 102 and the second screw element 104 prevents distraction along the longitudinal axis of the bone screw 100 but permits motion in one or more other degrees of freedom.

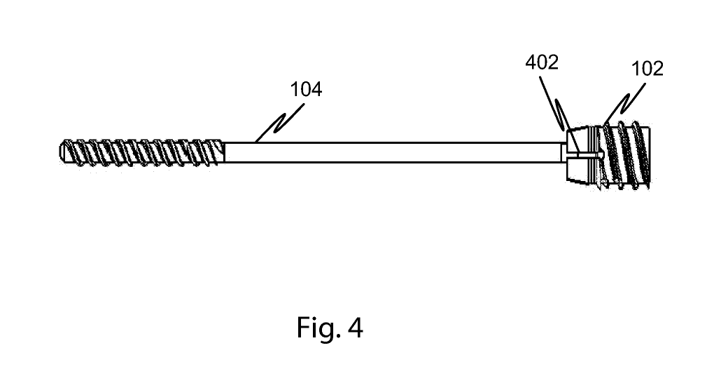

As shown in FIG. 1D, the engagement between the first screw element 102 and the second screw element 104 permits the second screw element 104 to be moved by an angle 118 of 0 to 15 degrees with respect to a coaxial alignment with the first screw element 102. Further, as shown in FIGS. 2A and 2B, the first screw element 102 allows the fastener head 202 to be recessed within the first screw element 102 to provide a smooth, low-profile implant. A smooth, low-profile implant can help reduce irritation to surrounding soft tissue. This example is explained in further detail in conjunction with FIGS. 4, 5, 6, and 7 below.

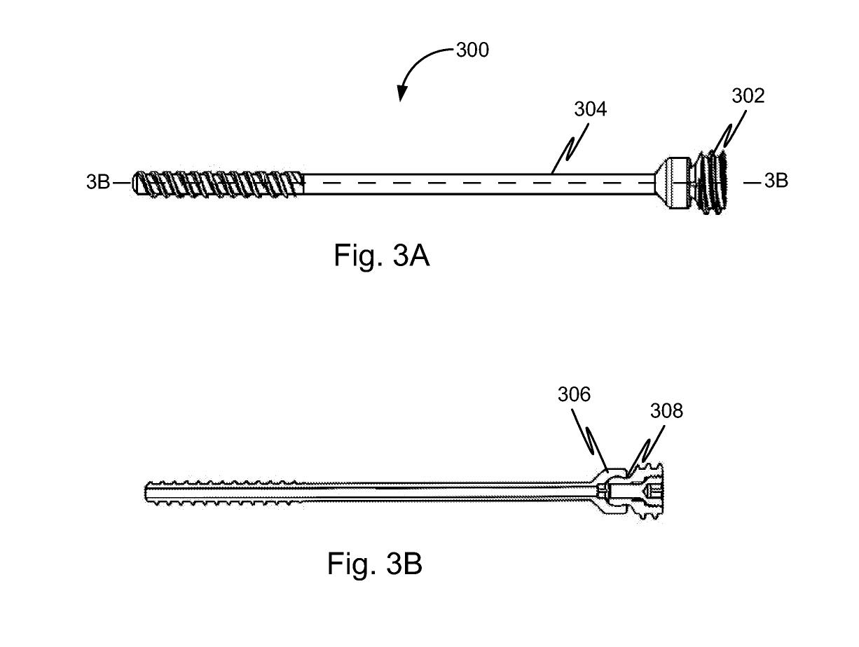

In a second example depicted in FIG. 3A and FIG. 3B, a bone screw 300 is shown comprising a first screw element 302 engaged with a second screw element 304. In this example, the second screw element 304 includes a partial spherical-shaped capsule 306 to receive a complementarily shaped, partial spherical fastener head 308 of the first screw element 302.

Referring back to FIG. 1B, the first screw element 102 also includes a stiffness reduction hole 120, near the distal end of the first screw element 102, such that the stiffness reduction hole 120 is placed on the side portion of the first screw element 102 for securely engaging the second screw element 104 with the first screw element 102. The first screw element 102 may include a plurality of stiffness reduction holes arranged in line with the stiffness reduction hole 120, on the circumference of the first screw element 102. Alternatively, as shown in FIG. 4A, the first screw element 102 may include one or more slits 402 on the distal end, such that the one or more slits 402 are aligned along the side portion of the first screw element 102 for securing the second screw element 104 with the first screw element 102.

Further, the first screw element 102 and the second screw element 104 may engage using one of a ball joint, a yoke joint, a universal joint, and a flat slider plate joint. Accordingly, the first screw element 102 and the second screw element 104 are suitably modified to engage with the used joint. Alternatively, a dumbbell element is placed between the first screw element 102 and the second screw element 104. The first screw element 102 and the second screw element 104 are suitably modified to receive one ball of the dumbbell element each, with a similar partial spherical-shaped capsule as described in conjunction with FIG. 2 above. The dumbbell element provides an additional set of degrees of freedom to the bone screw 100. Moreover, the first screw element 102 may be further secured to the second screw element 104 using a set screw.

Referring now to FIG. 5A, a second screw element 502 is shown with an external thread 504 and a partial spherical fastener head 506. FIG. 5B shows a cross section view along the line 5B-5B of FIG. 5A. FIG. 5C shows an enlarged view of a portion marked “5C” in FIG. 5B illustrating a hexalobe drive 508. FIG. 5D shows a top view of the second screw element 502 of FIG. 5A.

Referring now to FIG. 6A, a first screw element 602 is shown with an external thread 604 and a three-slot drive 606. FIG. 6B shows a top view of the first screw element 602 of FIG. 6A. FIG. 6C shows a side view of the first screw element 602. FIG. 6D shows a cross-section view along the line 6D-6D of FIG. 6C. A partial spherical-shaped capsule 608 is provided to receive the complementarily shaped, partial spherical fastener head 506 to obtain the bone screw of the present disclosure.

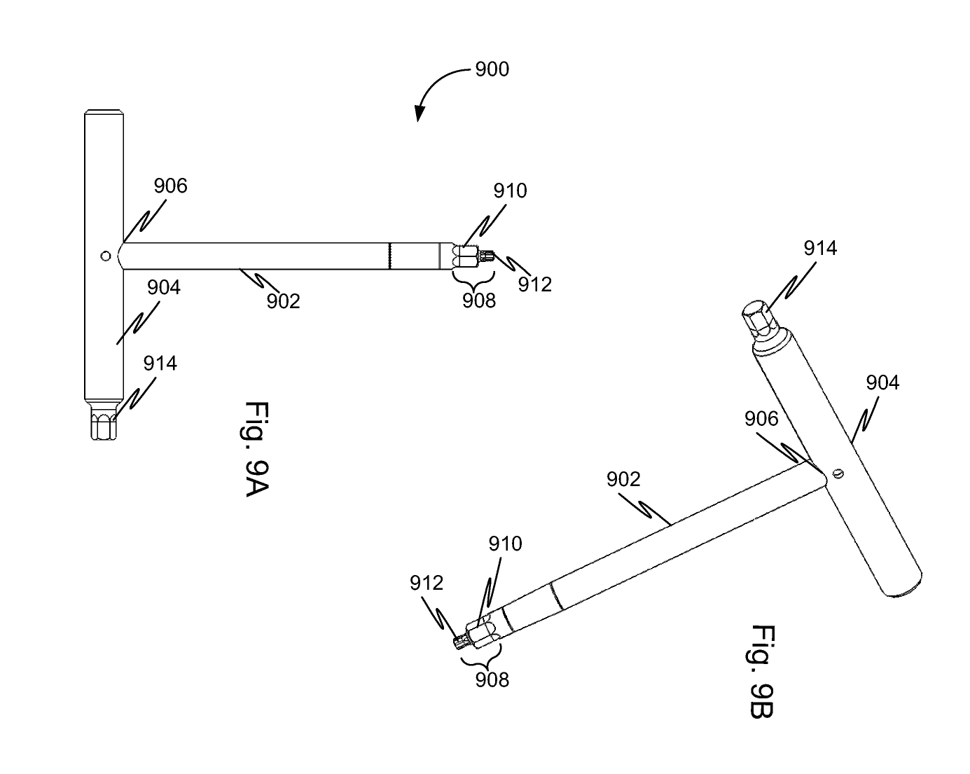

FIGS. 7A-7B, 8A-8B show two examples of drives that may be used in accordance with some examples of the present disclosure. FIG. 7A shows a cross-section view along a longitudinal axis of a bone screw 700, obtained after inserting a second screw element 702 through the first screw element 704 and engaged therewith. A partial spherical-shaped capsule 706 of the first screw element 704 receives a complementarily shaped, partial spherical fastener head 708 of the second screw element 702. Further, the first screw element 704 includes a three-slot drive 710. The second screw element 702 includes a hexalobe drive 712. FIG. 7B shows a top view of the bone screw 700. A stepped driver is used to simultaneously engage both the three-slot drive 710 and the hexalobe drive 712. A separate driver is used to engage only the three-slot drive 710 to selectively adjust the first screw element to achieve desired compression between bones.

FIG. 8A shows a cross-section view along a longitudinal axis of a bone screw 800, a obtained after inserting a second screw element 802 through a first screw element 804 and engaged therewith. FIG. 8B shows a top view of the bone screw 800. The first screw element 804 includes a stiffness reduction hole 806 on distal end of the first screw element 804. The stiffness reduction hole 806 allows for securely engaging the second screw element 802 with the first screw element 804. Further, the first screw element 804 includes a hex drive 808. The hex drive 808 may also include a tab 810 on each edge of the drive to allow a driver to engage with the first screw element 804. In addition, the tab 810 interacts with the driver to hold the first screw element in a specific orientation during insertion. The second screw element 802 includes a hexalobe drive 812.

Referring now to FIG. 9A showing a top view of a driver 900 that may be used to install the bone screw 800 in the bone. FIG. 9B shows an isometric view of the driver 900. The driver 900 includes two arms 902 and 904. One end of the arm 902 is partially inserted into a receptacle 906 in the side of the arm 904 and removably secured therewith. The other end of the arm 902 includes a stepped drive bit 908, comprising a first drive bit 910 that engages with the hex drive 808 and a second drive bit 912 that engages with the hexalobe drive 812. Further, one end of the arm 904 includes a drive bit 914 that engages only with the hex drive 808. A similar driver may be used for the bone screw 700, wherein instead of the first drive bit 910 and the drive bit 914, an alternative drive bit is used that engages with the three-slot drive 710.

A bone screw kit or system comprises a plurality of bone screws, wherein each bone screw includes a first screw element and a second screw element inserted through the first screw element and engaged therewith. The kit further includes at least one drill to provide clearance for both the first screw element and the second screw element in at least one bone, a first driver to drive the bone screw into the at least one bone, such that external thread on each of the first screw element and second screw element engages with the at least one bone, wherein the first driver is a stepped driver and the second driver selectively adjusts the first screw element to achieve a required compression.

Further, the at least one drill of the bone screw kit includes a cannulated major drill to provide clearance for the shaft of the second screw element, a cannulated minor drill to provide clearance for the threaded portion of the second screw element and a cannulated reamer to provide clearance for the first screw element.

Moreover, each bone screw in the kit is of a different configuration, wherein the configuration of a bone screw is defined by one or more parameters including a major diameter, a pitch, a length, a lag thread, and a drive connection. The bone screw kit may further include one or more of a K-wire, one or more tapping tools capable of accommodating all bone screws in the kit, and one or more driving tools.

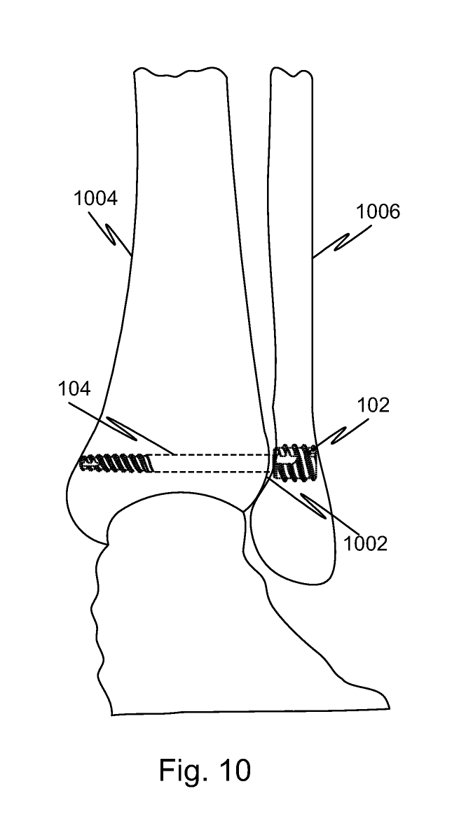

FIG. 10 shows an example of the placement of the bone screw 100 across a syndesmosis joint 1002 between a tibia 1004 and a fibula 1006. The shown placement of the bone screw 100 is easily achievable with the screw features, guides, drivers, and instrumentation disclosed herein. The external thread 112 of the second screw element 104 engages with the tibia 1004 and the external thread 106 of the first screw element 102 engages with the fibula 1006. The first screw element 102 may be made of PolyEther Ether Ketone (PEEK) and the second screw element 104 may be made of a titanium alloy.

As shown, the bone screw 100 when installed in the bones has a zero profile, which helps reduce irritation to surrounding soft tissue. Further, the fibula 1006 provides a measure of resistance to disengagement of the first screw element 102 and the second screw element 104. In addition, the joint between the first screw element 102 and the second screw element 104 allows for normal fibular motion, thereby reducing the chances of fracture of the bone screw 100. Further, the first screw element 102 may be selectively adjusted to fine-tune the syndesmosis compression.

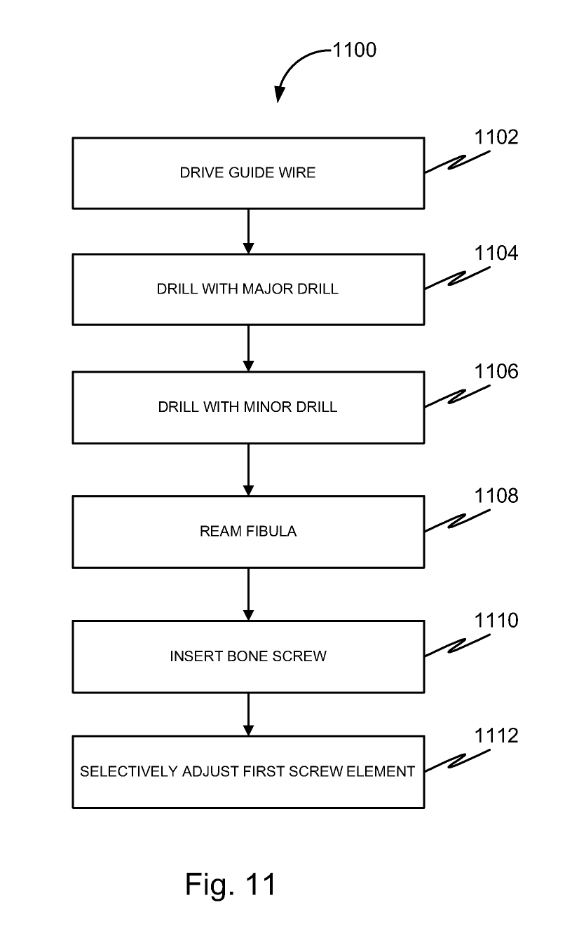

A method 1100 of inserting the bone screw 100 into the pair of bones, including a first bone and a second bone as disclosed herein, is described in FIG. 11. As shown in FIG. 10, the pair of bones includes the tibia 1004 and the fibula 1006. At step 1102, a surgeon inserts a guide wire (for example, a K-wire) to a desired location into the foot of a patient. In one example, the K-wire is inserted such that it reaches the fibula 1006. Once the guide wire is in the desired location, the surgeon may insert a dilator over the guide wire into the soft tissue of the patient to provide sufficient access to the bones. Once the tissue is dilated, the surgeon removes the dilator thus exposing the bones for the remainder of the surgery.

Next at step 1104, the surgeon guides a cannulated major drill over the guide wire to drill through the fibula 1006 and then through the tibia 1004. The major drill provides clearance for the shaft 108 (major diameter) of the second screw element 104. Then at step 1106, the surgeon uses a cannulated minor drill to provide clearance for threaded portion 110 (minor diameter). Further, at step 1108, the surgeon uses a fibular reamer to ream the fibula to provide clearance for the first screw element 102. The cannulated major drill, the cannulated minor drill and fibular reamer may be manually operated or may be operated by, or as, powered devices.

Once the implant site is sufficiently prepared to receive the bone screw 100, the guide wire is used to orient and insert the bone screw 100 into the fibula 1006 and the tibia 1004, at step 1110. The surgeon uses a first screwdriver that engages with both the first screw element 102 and the second screw element 104 to insert the bone screw 100 into the fibula 1006 and the tibia 1004. For example, if the bone screw 100 has a drive arrangement as shown in FIG. 8B, then the surgeon uses the stepped drive bit 908 of the driver 900 to engage with both the first screw element 102 and the second screw element 104. The self-tapping feature 114 and the self-tapping feature 116 tap the bones as the surgeon inserts the bone screw 100 into the fibula 1006 and the tibia 1004. The first screw element 102 engages with the fibula 1006 and the second screw element 104 engages with the tibia 1004.

Finally, at step 1112, the surgeon selectively adjusts the first screw element 102 to achieve a required compression between the bones. The surgeon uses a second screwdriver to selectively adjust the first screw element 102. The second screwdriver engages the first screw element 102 only. For example, if the bone screw 100 has a drive arrangement as shown in FIG. 8B, then the surgeon uses the driver bit 914 of the driver 900 to engage with the first screw element 102 only. Then, the surgeon may use a set screw to further secure the engagement of the first screw element 102 and the second screw element 104.

Once the bone screw 100 is in the proper location, the surgeon can remove the guide wire, the drivers, and any other instrumentation used, and then close the incision site.

It should be understood that the present components, systems, kits, apparatuses, and methods are not intended to be limited to the particular forms disclosed. Rather, they are intended to include all modifications, equivalents, and alternatives falling within the scope of the claims. They are further intended to include embodiments which may be formed by combining features from the disclosed embodiments, and variants thereof.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains,” one or more steps or elements, possesses those one or more steps or elements, but is not limited to possessing only those one or more elements. Likewise, a step of a method or an element of a device that “comprises,” “has,” “includes,” or “contains” one or more features, possesses those one or more features, but is not limited to possessing only those one or more features. Furthermore, a device or structure that is configured in a certain way is configured in at least that way, but may also be configured in ways that are not listed.

The present invention may be embodied in other specific forms without departing from its spirit or essential characteristics. It is appreciated that various features of the above-described examples can be mixed and matched to form a variety of other alternatives. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A bone screw, comprising:
a first screw element; and
a second screw element inserted through the first screw element and engaged therewith;
wherein each of the first screw element and second screw element including an external thread for threaded engagement with a bone, and wherein the first screw element is capable of being selectively adjusted to achieve a required compression.

2. The bone screw of claim 1, wherein the bone screw is used for fixation of the syndesmosis joint between the tibia and the fibula, such that the external thread of the second screw element engages with the tibia and the external thread of the first screw element engages with the fibula, wherein the first screw element is made of PolyEther Ether Ketone (PEEK) and the second screw element is made of a titanium alloy.

3. The bone screw of claim 1, wherein a pitch of the external thread on the first screw element is same as a pitch of the external thread on the second screw element.

4. The bone screw of claim 1, wherein the first screw element includes a three-slot drive and the second screw element includes a hexalobe drive, wherein a stepped driver is used to simultaneously engage both the three-slot drive and the hexalobe drive, wherein a three-slot driver is used to only engage the three-slot drive of the first screw element for selectively adjusting the first screw element to achieve the required compression.

5. The bone screw of claim 1, wherein the engagement between the first screw element and the second screw element permits the second screw element to be moved by an angle of about 15 degrees with respect to a coaxial alignment with the first screw element.

6. The bone screw of claim 1, wherein the first screw element and the second screw element engage using one of a ball joint, a yoke joint, a universal joint and a flat slider plate joint, and wherein the first screw element and the second screw element are suitably modified to engage with the used joint.

7. The bone screw of claim 1, wherein a dumbbell element placed between the first screw element and the second screw element engages the first screw element and the second screw element, and wherein the first screw element and the second screw element are suitably modified to receive one ball of the dumbbell element each.

8. The bone screw of claim 1, wherein the first screw element includes at least one slit on the distal end, wherein the at least one slit is aligned along the side portion of the first screw element for securing the second screw element with the first screw element.

9. The bone screw of claim 1, wherein the first screw element includes at least one stiffness reduction hole near the distal end of the first screw element, wherein the at least one stiffness reduction hole is placed on the side portion of the first screw element for securing the second screw element with the first screw element.

10. The bone screw of claim 1, wherein the first screw element is secured to the second screw element using a set screw.

11. The bone screw of claim 1, wherein the second screw element is cannulated.

12. The bone screw of claim 1, wherein the second screw element has a lag thread configuration.

13. The bone screw of claim 1, wherein the first screw element includes a partial spherical-shaped capsule to receive a complementarily shaped partial spherical fastener head of the second screw element.

14. A method of fixing a joint between a pair of bones, the method comprising:
providing a bone screw, comprising
a first screw element; and
a second screw element inserted through the first screw element and engaged therewith;
wherein each of the first screw element and second screw element include an external thread for threaded engagement with the pair of bones;
driving the bone screw into the pair of bones such that an external thread of each screw element is disposed in threaded engagement with a different bone; and
selectively adjusting the first screw element to achieve a required compression.

15. The method of claim 14, wherein driving the bone screw into the pair of bones including a first bone and a second bone, further comprises:
driving a Kirschner-wire to a desired position;
using a cannulated major drill to provide clearance for the shaft of the second screw element in the first bone;
employing a cannulated minor drill to provide clearance for the threaded portion of the second screw element in the first bone;
utilizing a cannulated reamer to provide clearance for the first screw element in the second bone; and
inserting the bone screw through the first bone and the second bone using a first screwdriver that engages both the first screw element and the second screw element.

16. The method of claim 14, wherein selectively adjusting the first screw element further comprises using a second screwdriver to achieve the required compression between the pair of bones, wherein the second screwdriver engages the first screw element only.

17. The method of claim 14, wherein the pair of bones includes the tibia and the fibula, and wherein the external thread of the second screw element engages with the tibia and the external thread of the first screw element engages with the fibula.

18. A bone screw system, comprising:
a plurality of bone screws, wherein each bone screw includes a first screw element and a second screw element, wherein the second screw element is inserted through the first screw element and engaged with the first screw element;
at least one drill to provide clearance for the both first screw element and the second screw element in at least one bone;
a first driver to drive the bone screw into the at least one bone, such that external thread on each the first screw element and second screw element engages with the at least one bone, wherein the first driver is a stepped driver; and
a second driver to selectively adjust the first screw element to achieve a required compression.

19. The bone screw system of claim 18, wherein the at least one drill includes:
a cannulated major drill to provide clearance for the shaft of the second screw element;
a cannulated minor drill to provide clearance for the threaded portion of the second screw element; and
a cannulated reamer to provide clearance for the first screw element.

20. The bone screw system of claim 18, wherein each bone screw in the system is of a different configuration, wherein the configuration of a bone screw is defined by one or more parameters including a major diameter, a pitch, a length, a lag thread and a drive connection, and wherein the bone screw system further comprising one or more of a K-wire, one or more tapping tools capable of accommodating all bone screws in the system, and one or more driving tools.
